(19) United States
(12) Patent Application Publication
(10) Pub. No.: US 2024/0041377 A1
(43) Pub. Date: Feb. 8, 2024

(54) FLAT TEXTILE MATERIAL FOR PLACING ON A HUMAN OR ANIMAL BODY

(71) Applicant: KOB GMBH, Wolfstein (DE)

(72) Inventors: Sina Borczyk, Kaiserslautern (DE); Pulkit Mishra, Otterbach (DE); Ferdinand Tamoué, Ludwigshafen am Rhein (DE); Marcin Meyer, Wolfstein (DE)

(21) Appl. No.: 18/268,700

(22) PCT Filed: Dec. 15, 2021
(86) PCT No.: PCT/EP2021/085807

§ 371 Date: Jun. 21, 2023

(30) Foreign Application Priority Data
Dec. 22, 2020 (DE) 10 2020 134 698.0

(51) Int. Cl. A61B 5/27; A61B 5/00; A61F 13/00

(52) U.S. Cl. CPC A61B 5/27; A61B 5/445; A61B 5/4848; A61F 13/00042; A61F 13/00029; A61F 2013/8473

(57) ABSTRACT

The invention relates to a flat textile material (2) for placing on a human or animal body in order to capture and/or transmit electrical signals, comprising: a longitudinal direction (4); a traverse direction (6) running perpendicular to the longitudinal direction (4); an extension plane (8); a thickness direction (10) orthogonal to the extension plane (8); a first layer (34) facing the body; a second layer (36) facing away from the body; and at least one electrode (20), arranged on or in the first layer (34), for placing on the body, wherein: the electrode (20) comprises electrically conductive threads (50, 52); the second layer (36) comprises electrically non-conductive threads (56, 58) having a first water absorbency; threads (64) extending in the longitudinal direction (4) and having a second water absorbency are provided either in the first layer (34) or in the thickness direction (10) between the electrode (20) and the second layer (36); and the second water absorbency of these threads (64) is greater than the first water absorbency of the electrically non-conductive threads (56, 58) of the second layer (36).

The invention relates to a flat textile material for placing on a human or animal body in order to capture and/or transmit electrical signals.

It is already known, for example, from DE 10 2017 126 463 A1 to use textile-based contact electrodes for the transcutaneous transmission of electrical signals, wherein the electrode comprises an electrically conductive textile and an electrically conductive polymer, wherein the electrically conductive textile is partially penetrated by the electrically conductive polymer. The electrode is held in a layer of flat textile material.

The object underlying the present invention is to provide a flat textile material of the mentioned type which, including the electrode, is textile-based, that is to say can be produced by a textile method, and which can be used for capturing and transmitting electrical signals originating from the body or to the body. Such signals are typically also understood as being biophysiological potentials which are captured at the body, but also potentials that stimulate the body, which are thus conducted to the body.

The flat textile material is preferably to be suitable both for long-term use and for multiple usage. In particular, the flat textile material could also be used to assist and monitor wound treatment and wound management, namely in particular integrated in a wound covering or a wound dressing in the broad sense. However, the flat textile material could also be used in physiological or medical measuring devices of any kind which are designed for use on the human or animal body.

In order to achieve this object there is proposed according to the invention a flat textile material having the features of claim 1.

It is hereby possible on the one hand to produce an electrode from or having electrically conductive threads which is capable of being produced in a textile process. In particular, the electrode can be configured as a region of the first layer which is formed by the conductive threads and which can additionally comprise further threads, in particular electrically non-conductive threads. Because there is present at least in the region of the electrode a second layer, facing away from the body, having electrically non-conductive threads having the first relatively lower water absorbency, the electrode can be covered and thus protected on the side facing away from the body by the second layer, and, as a result of the first relatively lower water absorbency of the threads of the second layer, in particular as a result of a hydrophobic configuration of this second layer, protection against the ingress of excessive moisture to the electrode and also evaporation protection, that is to say protection against drying out of the body region and of the flat textile material in the region of the electrode, can be achieved, so that the functionality thereof is maintained and supported.

By means of the further threads having the second relatively higher water absorbency which run in the longitudinal direction in the first layer or between the electrode and the second layer, on the one hand excessive moisture or even liquid in the electrode region can be absorbed, wherein this does not, however, lead to drying out but rather to moisture retention and conditioning of the electrode region. Because these threads having the second relatively higher water absorbency are preferably arranged not in direct contact with the skin but where possible on the side of the electrode facing away from the body, they do not lead to exsiccation of the area of the skin with which the electrode is in direct contact, but instead it is assumed, on the contrary, that as a result of these threads, in particular assisted by a contact pressure, a moisture conditioning of the electrode can take place from the side facing away from the body, whereby an electrical contact resistance between the skin and the electrode can be reduced, which leads to good contacting.

The water absorbency of threads can be determined in accordance with standard DIN 53923:1978-01. Instead of the flat test specimens mentioned in the standard, thread portions having a length of 80 mm are used. At least 1 g of the thread portions are used as the sample for each measurement. Alternatively, it is also possible to use a continuous thread which is sufficiently long that it has a mass of at least 1 g. The sample adjusted to a standard reference atmosphere according to DIN 53802 is weighed to the nearest 0.01 g (mass dry mt). The thread portions or the thread are then arranged on a wire gauze side by side, or in the case of the continuous thread going back and forth, and fastened at their ends with clamps. The wire gauze with the sample placed thereon is placed in a dish containing distilled water at (20+/−1) ° C., so that the sample is about 20 mm beneath the water level. After an exposure time of approximately 60 seconds, the wire gauze with the sample is removed and, after the removal of some clamps, held for approximately 2 minutes in such a way that the thread portions, hanging freely, are able to drip. The sample is then removed from the wire gauze, placed into a weighing bottle and again weighed to the nearest 0.01 g (mass wet mf). The water absorbency W_{A} is determined in accordance with the following easily comprehensible calculation and given in wt. %:

W_{A}=((mass wet−mass dry)/mass dry)*100

W_{A}=((mf−mt)/mt)*100.

It is here found to be advantageous if the second water absorbency is at least 1.10 times, in particular at least 2.0 times and further in particular at least 5.0 times, in particular at least 10 times, in particular at least 20 times, the first water absorbency (claim 2).

It is further found to be advantageous if the first water absorbency is not more than 1.0 wt. %, in particular not more than 0.1 wt. %, in particular not more than 0.01 wt. %. This means that the thread portions are able to absorb not more than 1.0% or not more than 0.1% or not more than 0.01% of their mass of water (claim 3).

It is further found to be advantageous if the second water absorbency is at least 2.0 wt. %, in particular at least 5.0 wt. %, in particular at least 20 wt. %. This then means that the thread portions are able to absorb at least 2.0% of their mass of water (claim 4).

Where mention is made here of electrically conductive threads, this means that such an electrically conductive thread comprises an electrically conductive material, in particular is coated therewith, or consists of an electrically conductive material. For example, a thread of any kind can have a metallic and thus electrically conductive coating, by which electrical conductivity of the thread is brought about. A thread that is considered to be electrically conductive has an electrical resistance of not more than 2000 ohms/m, preferably of not more than 1000 ohms/m, preferably of not more than 500 ohms/m. This means that a piece of thread having a length of 1 m, when subjected to a conventional resistance measurement, has an electrical resistance of not more than 2000 ohms or not more than 1000 ohms or not more than 500 ohms.

An electrically non-conductive thread generally at least does not comprise any typical electrically conductive materials, such as metals, and in particular does not have a metallic coating. An electrically non-conductive thread, in accordance with the logic of physics, has a very high electrical resistance approaching infinity. However, a thread can be considered to be electrically non-conductive if it has an electrical resistance of more than 10⁶ ohms/m.

In order to test whether a thread is electrically conductive or electrically non-conductive, threads conditioned under laboratory standard conditions are used, that is to say threads which are dry and which have been conditioned for at least 24 hours at a temperature of 20° C. and a relative humidity of 65%.

The flat textile material is advantageously configured in that the electrically conductive threads of the electrode comprise electrically conductive threads extending in the longitudinal direction and electrically conductive threads extending in the transverse direction (claim 5).

As a result, the electrode can advantageously be produced in the form of a textile structure, in particular in the form of a woven fabric.

It is further found to be advantageous if the electrically non-conductive threads of the second layer comprise electrically non-conductive threads extending in the longitudinal direction and electrically non-conductive threads extending in the transverse direction, which threads in each case have the first water absorbency (claim 6).

It can be found to be advantageous in particular if the second layer consists of electrically non-conductive threads, in particular of threads which each have the first water absorbency.

It can be found to be advantageous in particular if the electrode consists of electrically conductive threads.

The flat textile material can further be so configured that there is at least one side region adjoining the electrode in the transverse direction, in particular a first side region adjoining the electrode in the transverse direction and a second side region adjoining the electrode in the transverse direction opposite the first side region (claim 7). In this embodiment, the electrode of the flat material is thus flanked in the transverse direction on one side or on both sides by the side region. The side region of the flat material is formed by the first layer and/or by the second layer, that is to say the first layer and/or the second layer are extended or present there over a wide area.

According to a further embodiment of the flat material according to the invention, it is provided that the electrode or the electrode and the side region(s) form an electrode portion of the flat material, and that the electrode portion is adjoined in the longitudinal direction by an intermediate portion of the flat material (claim 8).

An alternative embodiment can further be so configured that the electrode portion is adjoined in the longitudinal direction by a further intermediate portion opposite the intermediate portion (claim 9). In this embodiment, an arrangement of intermediate portion-electrode portion-intermediate portion is thus obtained in the flat textile material in the longitudinal direction.

It can also be found to be advantageous that the intermediate portion is adjoined in the longitudinal direction by a further electrode portion, wherein the intermediate portion is then arranged in the longitudinal direction between the electrode portions (claim 10). In this last embodiment, an arrangement of electrode portion-intermediate portion-electrode portion is thus obtained in the flat textile material in the longitudinal direction.

It can, however, also be found to be particularly advantageous if the flat textile material is configured to be substantially endless in the longitudinal direction, wherein an electrode portion and an intermediate portion alternate (claim 11).

With regard to continuous textile producibility, it is found to be advantageous if the first layer comprises in the side region(s) of the electrode portion electrically non-conductive threads extending in the longitudinal direction and electrically conductive threads extending in the transverse direction (claim 12). This makes it possible for the first layer to be formed in the transverse direction outside the actual electrode region at least also by the same conductive threads extending in the transverse direction as form the electrode in the actual electrode region, that is to say in the transverse direction between the side regions.

It is further found to be advantageous if the threads having the second relatively higher water absorbency extending in the longitudinal direction are arranged in the transverse direction between the first side region and the second side region (claim 13). This means that these threads are preferably present in the transverse direction only in the region of the electrode, that is to say do not run in the transverse direction outside the actual electrode region.

In a further embodiment, it is found to be advantageous if these threads having the second relatively higher water absorbency are provided in a floating manner between the electrode and the second layer (claim 14).

This means that in the region of the electrode these threads are not integrated in a textile manner into the first and second layers but instead run between the layers in particular extended substantially parallel to one another. In this manner, these more hydrophilic threads are able to develop their moisture-conditioning action unhindered by a textile weave.

It is further found to be advantageous if the first layer and the second layer lie loosely against or on one another at least in the electrode portion (claim 15). As a result of this further measure too, a gap is created between the first and the second layers, in which the above-mentioned threads having the second water absorbency are able to extend and, depending on the amount of liquid or moisture, are able to absorb more or less moisture and thereby swell.

It can also be found to be advantageous if the first layer and the second layer are joined together in the intermediate portion (claim 16).

In particular, it can further be found to be advantageous if the first layer and the second layer are brought together in the intermediate portion to form a combined textile carrier layer (claim 17). If the flat textile material—as mentioned at the beginning—has an electrode portion and an intermediate portion following one another and in particular alternating endlessly in the longitudinal direction, then multi-layer electrode portions and single-layer intermediate portions alternate. This has the advantage that the two layers form a stable textile carrier layer in the intermediate portion, and that in the electrode portion the first and the second layers are separated from one another and in particular lie against one another in the thickness direction, as has also already been mentioned hereinbefore.

In a further development of the flat material according to the invention, it is proposed that the threads having the second water absorbency in the intermediate portion are configured and arranged to be placed on the body (claim 18). This allows these relatively more hydrophilic threads in the intermediate layer to purposively be used for the exsiccation of the surface of the body. In the intermediate portion, that is to say in the region in the longitudinal direction between the electrodes, a moist body surface is also disadvantageous because it is metrologically desirable to electrically insulate the regions between two measuring electrodes from one another where possible, that is to say to prevent an electrical short circuit between the electrodes as a result of an electrically conducting surface connection by way of a moist body surface.

According to a further idea which is of particular importance, it is found to be advantageous if the electrically conductive threads of the electrode that are arranged in the longitudinal direction pass through the second layer and/or the carrier layer in the thickness direction and are arranged preferably in a floating manner on the side of the second layer or of the carrier layer facing away from the body (claim 19). If these electrically conductive threads running in the longitudinal direction extend endlessly in the direction of manufacture of the flat textile material, that is to say in the longitudinal direction, then they can also form the respective electrode following in the longitudinal direction. In such a case, an endless manufacturing process for producing the flat textile material can thus be implemented.

It is possible to cut these electrically conductive threads of the electrode that run in the longitudinal direction in the region between two successive electrodes and preferably combine the respective cut ends to form a bundle (claim 20). An electrical terminal can thus be formed. It would, however, also be conceivable to cut the electrically conductive threads in particular close to the electrode and to discard an intermediate piece thereby produced. On the other hand, should it be desired in some applications to keep electrodes arranged next to one another connected together in an electrically conducting manner as contact regions, then separation, that is to say capping, of the threads would not be carried out.

According to a further embodiment, it is found to be advantageous that the intermediate portion comprises a middle region, wherein the middle region has a transverse extent in the transverse direction that corresponds substantially to the electrode, wherein there are provided in the middle region threads having the second water absorbency which extend in the longitudinal direction and are arranged side by side substantially in parallel and electrically non-conductive threads having the first water absorbency which extend in the longitudinal direction and are arranged side by side substantially in parallel (claim 21).

In the transverse direction outside a middle region, the intermediate portion is formed in particular by electrically non-conductive threads extending in the longitudinal direction and by electrically conductive and/or electrically non-conductive threads extending in the transverse direction (claim 22).

In a further particularly advantageous embodiment, it is provided that the first layer and the second layer are arranged and joined together such that there is formed in the electrode portion a tunnel which extends in the transverse direction and is delimited by the first and second layers (claim 23).

This opens up the possibility not only of providing this tunnel, that is to say the gap between the first and second layers, as an extension space for the above-mentioned threads having the second water absorbency, but also of providing an additional space for functional elements. In particular, it is conceivable and advantageous that a pressure element can be inserted into the tunnel, which pressure element urges the first layer and the electrode, and optionally the more hydrophilic threads having the second water absorbency which are guided transversely through the tunnel in a floating manner, toward the body (claim 24). By the use of an additional element, a contact pressure can thus be achieved, which presses the electrode against the surface of the body, which assists with the formation of good electrical contact. The receiving space formed by the tunnel can, however, also be used to receive electrical components, in particular amplifier components or electrical or electronic measuring or amplifier or control devices of any kind.

It is further proposed that the electrode is configured to be raised relative to the body as compared to the side regions and/or as compared to the intermediate portion and/or as compared to the carrier layer (claim 25). This can be achieved, for example, by providing a larger number of threads extending in the longitudinal direction and/or in the transverse direction in the actual electrode region than in the adjoining regions outside the electrode. It is, however, also conceivable that this can be achieved by the alternating multi-layer and single-layer configuration already mentioned.

Where mention is made hereinbefore of a flat textile material and of threads running in the longitudinal direction and in the transverse direction, the specific type of textile weave is not necessarily specified, as long as threads extend substantially in the longitudinal direction and transverse thereto, which has hitherto not been meant in the sense of a geometrically strictly linear extent. However, protection is preferably claimed for a flat textile material in which the first and/or the second layer is formed by a woven fabric, wherein a warp direction of the woven fabric runs in the longitudinal direction and a weft direction of the woven fabric runs in the transverse direction (claim 26). In this case, above-mentioned threads extended in the longitudinal direction are warp threads and above-mentioned threads extended in the transverse direction are weft threads of the woven fabric.

The invention also provides a wound dressing for assisting and monitoring wound treatment, which comprises a flat textile material according to the invention (claim 27).

Preferably, the flat textile material as a whole can be in the form of a woven fabric formed of warp threads and weft threads. The warp threads are preferably supplied endlessly in a production direction of the flat textile material. The weft threads extended transverse thereto can end freely at lateral longitudinal edges of the flat textile material, or an edging of any kind, in particular in the form of a hem, can be created there. It is also possible to provide the transverse edges or longitudinal edges of a portion of the flat textile material with an edging of any kind, in particular with a hem.

The invention is further considered to be independently substantiated by:

A flat textile material (2) for placing on a human or animal body in order to capture and/or transmit electrical signals, comprising:
a longitudinal direction (4),
a transverse direction (6) running transverse to the longitudinal direction (4),
an extension plane (8),
a thickness direction (10) orthogonal to the extension plane (8),
a first layer (34) facing the body,
a second layer (36) facing away from the body,
at least one electrode (20) arranged on or in the first layer (23) for placing on the body,
wherein the electrode (20) comprises electrically conductive threads (50, 52),
wherein the second layer (36) comprises electrically non-conductive threads (56, 58), and
wherein there can optionally be present at least one first and/or second side region (26, 28) adjoining the electrode (20) in the transverse direction (6),
wherein the first layer (34) facing the body forms the electrode and the side region(s) (26, 28) optionally present,
wherein the electrode (20) and the optional side region(s) 26, 28) form an electrode portion (30) of the flat material (2), and
wherein the electrode portion (30) is adjoined in the longitudinal direction (4) by an intermediate portion (32) of the flat material (2),
wherein the first layer (34) and the second layer (36) lie loosely against or on each other in the electrode portion (30), and
wherein the first layer (34) and the second layer (36) are brought together in the intermediate portion (32) to form a combined textile carrier layer (42).

In a further development of this subject-matter, the features of one or more of the dependent patent claims can additionally be provided.

It can also be found to be advantageous in the case of this subject-matter if there are provided either in the first layer (34) or in the thickness direction (10) between the electrode (20) and the second layer (36) threads (64) having a water absorbency of at least 2.0 wt. %, in particular of at least 5.0 wt. %, in particular of at least 20 wt. %, extending in the longitudinal direction (4).

It can further be found to be advantageous if the water absorbency of those threads (64) is higher than and in particular at least 1.10 times, in particular at least 2.0 times and further in particular at least 5.0 times, in particular at least 10 times, in particular at least 20 times, a water absorbency of the electrically non-conductive threads of the second layer (36).

Further features, details and advantages of the invention will become apparent from the accompanying patent claims and from the representation shown in the drawing and the following description of a preferred embodiment of the flat textile material according to the invention.

In the drawing:

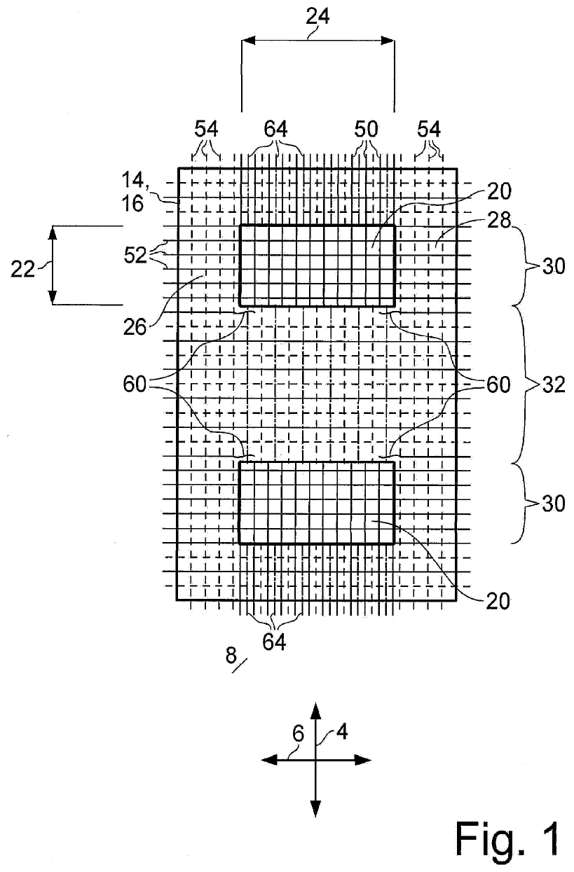

FIG. 1 is a schematic plan view of a side of a flat textile material according to the invention facing the skin;

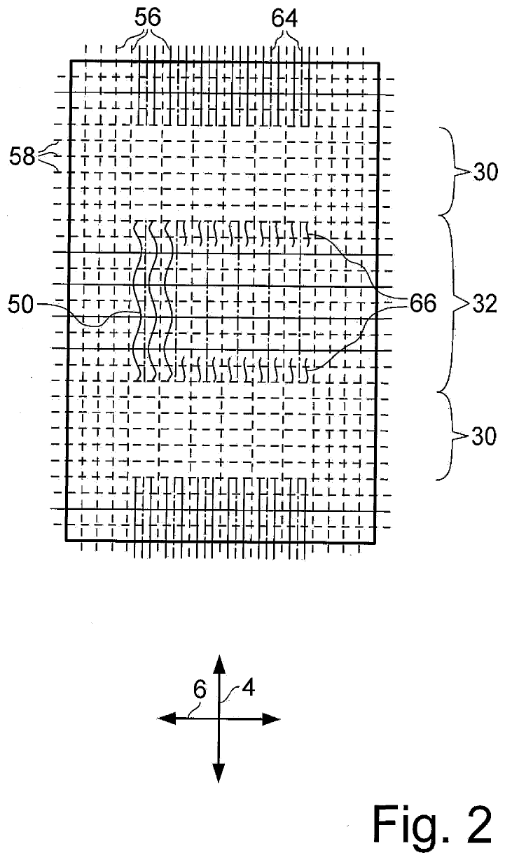

FIG. 2 is a schematic plan view of a side of the flat material according to FIG. 1 facing away from the skin;

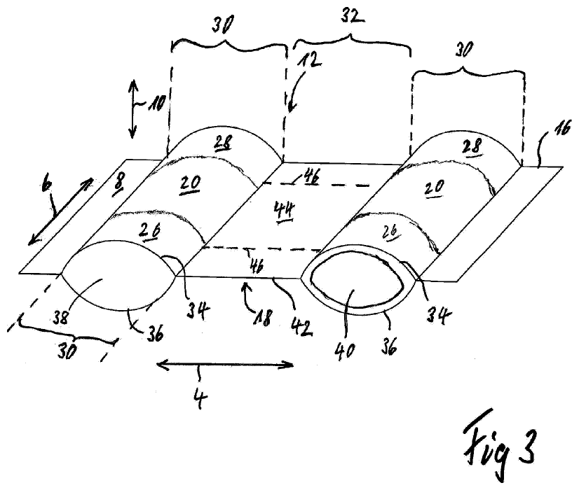

FIG. 3 is a schematic three-dimensional representation of a portion of a textile flat material according to the invention looking from above at a side facing the body;

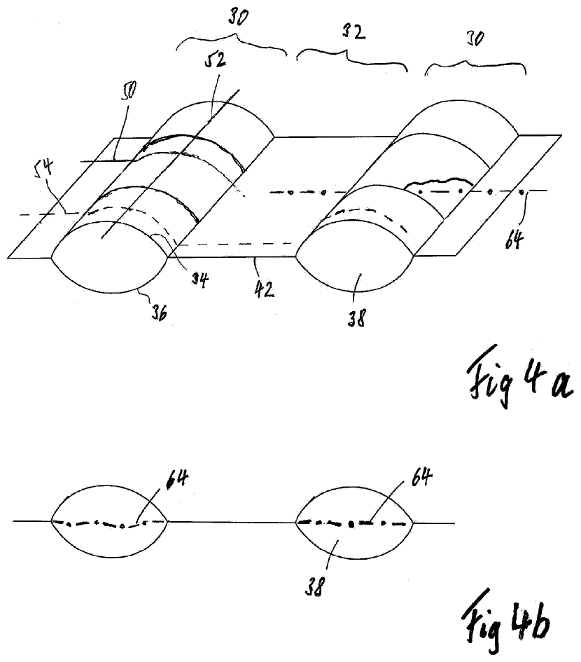

FIG. 4a is a further representation corresponding to FIG. 3; and

FIG. 4b is a schematic side view of the portion according to FIG. 3;

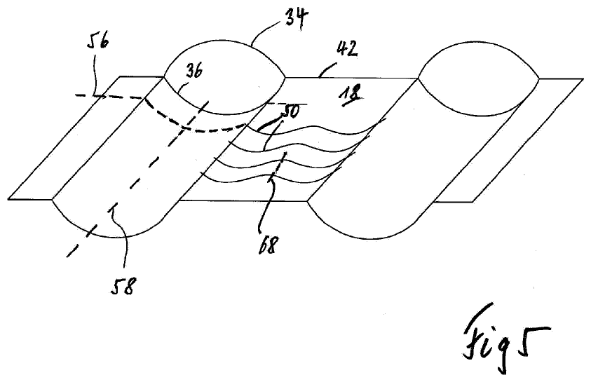

FIG. 5 is a further schematic representation of the portion according to FIG. 3, looking from beneath at a side facing away from the body;

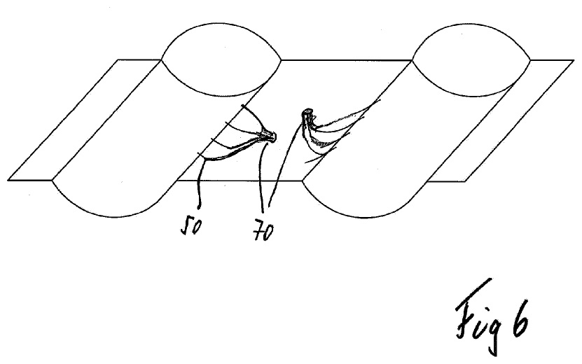

FIG. 6 is a further schematic representation corresponding to FIG. 5 with cut and bundled electrically conductive threads;

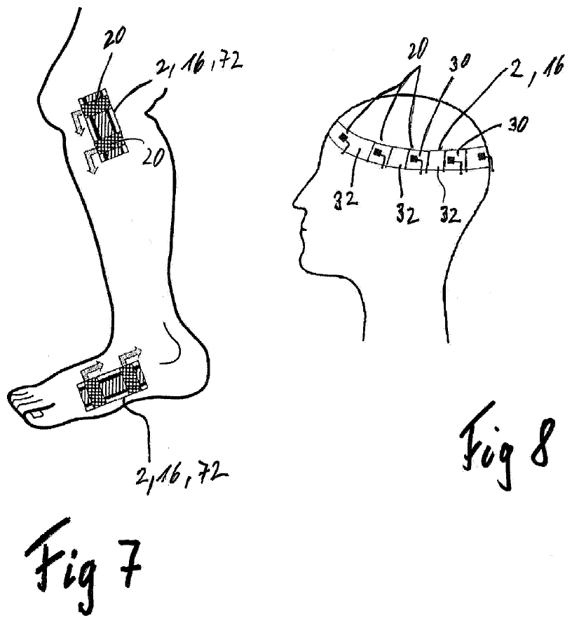

FIG. 7 is a representation of a body part with two portions of a flat textile material according to the invention placed on the body part, in an operating situation; and

FIG. 8 is a further representation of a body part with an annular closed band of a flat textile material according to the invention having a plurality of electrode portions and intermediate portions.

The figures show a flat textile material 2 according to the invention and illustrate, in some cases highly schematically, the structure thereof. The flat material 2 is intended to be placed on a human or animal body and to capture electrical signals and/or transmit them to the body. The flat textile material 2 extends in a longitudinal direction 4 and in a transverse direction 6 and in an extension plane 8 which coincides with the plane of the drawing of FIG. 1. A thickness direction 10 running orthogonal to the extension plane 8 is further inserted, and this is visible in FIG. 3.

FIG. 1 is a plan view of a side 12, facing the body, of a portion 16 of the flat textile material 2 indicated by a rectangular frame 14. FIG. 2 is a plan view of a side 18 of the portion 16 facing away from the body. In the highly schematic perspective representation of the flat material portion 16 according to FIG. 3, the side 12 facing the body is at the top facing the viewing direction and the side 18 facing away from the body is at the bottom facing away from the viewing direction.

In the case shown here, the portion 16 of the flat textile material 2 comprises by way of example two electrodes 20 for capturing or transmitting electrical signals, which electrodes are by way of example rectangular and are indicated in FIG. 1 by a frame 21. They extend over an electrode length 22 in the longitudinal direction 4 and over an electrode width 24 in the transverse direction 6. In the transverse direction 6, outside each electrode 20, there follows a first and a second side region 26, 28 of the portion 16. Each side region 26, 28 is also extended over the electrode length 22 and in the transverse direction 6 in each case as far as the edge of the flat textile material 2. The region of each electrode 20, together with the first and the second side regions 26, 28, forms in each case an electrode portion 30 of the flat textile material 2, or of the portion 16. In the longitudinal direction 4 between the electrode portions 30, an intermediate portion 32 of the flat textile material 2 is provided or arranged. In this intermediate portion 32, the two electrodes 20 are in most applications to be largely electrically separated in the longitudinal direction 4.

As will be explained in detail, the flat textile material 2 according to the invention can be produced endlessly in the longitudinal direction 4, so that the flat material portions 16 indicated in the figures can be obtained from an endlessly produced strip or band of the flat material according to the invention in the form of longitudinal portions of that endless flat material 2. It is thus conceivable in particular that a portion 16 of the flat material 2 in question can also have a plurality of electrodes 20, between which there are arranged in the longitudinal direction 4, as shown, intermediate portions 32 which electrically separate the electrodes 20 from one another as far as possible or, for certain applications, leave them electrically connected to one another.

As can be seen from the highly schematic representation of the portion 16 of the flat textile material 2 in FIG. 3, the flat textile material 2 comprises in each electrode portion 30 a first layer 34 facing the body and a second layer 36 facing away from the body, which layers in each electrode portion 30 can in particular, in the preferred case shown, be loosely placed against one another and/or, as shown in an exaggerated manner in FIG. 3, can be opened up and then delimit a tunnel 38 extended in the transverse direction 6. The tunnel 38 can preferably extend over the entire transverse extent of the flat material portion 16, that is to say delimit a continuous clear cross-sectional area into which, for example, a pressure element indicated on the right in FIG. 3 can be inserted in order to urge or press the electrode 20 of the flat material 2 in the direction toward the body, in order to assist with achieving good electrical contacting of the electrode 20 with the surface of the body. This is optional, however, and the tunnel 38 can also remain unfilled during use and the layers 34, 36 can to a certain extent lie loosely against one another. If desired, they could also be joined together in any desired manner, for example by means of stitching.

As is likewise best apparent from FIG. 3, the first layer 34 facing the body and the second layer 36 facing away from the body are brought together in the intermediate portion 32 to form a combined textile carrier layer 42, which will be explained in greater detail below. At the transition from the intermediate portion 32 into the adjoining further electrode portion 30, the threads of the textile carrier layer 42 are then separated again in the thickness direction 10 in order to form there the first layer 34 facing the body and the second layer 36 facing away from the body.

Finally, a middle region 44 is also defined in the intermediate portion 32, which middle region is situated in the transverse direction 6 between two broken lines 46 and the transverse extent of which corresponds approximately to the electrode width 24.

The formation of the layers, of the electrode, of the regions and portions of the flat textile material 2 according to the invention will now be explained.

Each electrode 20 comprises electrically conductive threads 50 extended in the longitudinal direction 4 and electrically conductive threads 52 extended in the transverse direction 6. In the case shown by way of example, these electrically conductive threads 50, 52 form both the electrode 50 and the first layer 34 facing the body in the region of the electrode.

The course of a single electrically conductive thread 50 in the form of a warp thread and 52 in the form of a weft thread is shown schematically in FIGS. 4 and 5. FIG. 4 shows the flat material portion 16 in perspective and in the viewing direction of FIG. 3, and FIG. 5 shows the flat material portion 16 from beneath, looking at the side 18 of the portion 16 facing away from the body. In FIG. 5, looking at the side 18 facing away from the body, a plurality of electrically conductive threads 50 running in a wavy line in the longitudinal direction 4 are therefore shown, in order to illustrate a floating, that is to say unwoven, course of these threads in the intermediate portion 32. At the transition from the electrode portion 30 into the intermediate portion 32, each thread 50 thus passes in the thickness direction 10 through the second layer 36 facing away from the body or through the carrier layer 42 in the intermediate portion 32 and, as indicated in FIG. 5, continues to run in the longitudinal direction 4 in a floating manner and to a certain extent parallel to the carrier layer 42, which is yet to be explained.

In the transverse direction 6 outside the electrode 20, that is to say in side regions 26, 28 of the flat material 2, the first layer 34 facing the body is formed by electrically non-conductive threads 54 running in the longitudinal direction 4 and by the electrically conductive threads 52 running in the transverse direction 6, which also form the electrode 20. Further threads could be present, in particular in the longitudinal direction 6, but this is not the case in the example.

In the case shown by way of example, the second layer 36 facing away from the body is formed in the region of the electrode portion 30 of the flat textile material 2 by electrically non-conductive threads 56 extending in the longitudinal direction 4 and by electrically non-conductive threads 58 extending in the transverse direction 6. The threads 56 extended in the longitudinal direction, as warp threads, form a woven fabric with the threads 58 extended in the transverse direction 6, as weft threads. Here too, a single thread 56 and 58 is indicated in FIG. 5 for the purpose of illustration.

The threads 56, 58 have a first water absorbency, as explained at the beginning. The second layer 36 can therefore be described as more rather hydrophobic, so that it prevents or stops the ingress of liquid or excessive moisture from outside through the second layer 36 in the direction toward the electrode 20.

In the case shown by way of example, the first layer 34 facing the body and the second layer 36 facing away from the body merge into each other in the intermediate portion 32 to form the combined textile carrier layer 42. This carrier layer 42 accordingly comprises in the intermediate portion 32 the electrically conductive threads 52 extended in the longitudinal direction and the electrically non-conductive threads 54 extended in the longitudinal direction and the non-conductive threads 58 extended in the transverse direction, which are present not only in the electrode portion 30 but also in the intermediate portion 32. As is indicated in FIG. 1 at the transition of the electrode portion 30 into the intermediate portion 32, some adjacent electrically conductive threads 52 running in the transverse direction have been cut on both sides (see reference numeral 60), in order to achieve electrical insulation of the electrode 30 from the textile carrier layer 42 in the intermediate portion 32.

The flat textile material 2 comprises further threads 64 extended in the longitudinal direction 4, but these run in the longitudinal direction 4 along the entire flat textile material only in a middle region, which corresponds approximately to the electrode width 24. These threads 64 run in the electrode portion 30, by way of example and preferably, in a floating manner, that is to say they run in the longitudinal direction 4 between the first layer 34 facing the body and the second layer 36 facing away from the body and to a certain extent pass through the tunnel 38 formed by those layers. They are then woven in in the intermediate portion 32, that is to say in the textile carrier layer 42, as further warp threads. For further illustration, a single one of these threads 64 is shown in FIG. 4a in the right-hand half and a plurality of these threads 64 is shown in FIGS. 1 and 2 as a dot-and-dash line. FIG. 4b illustrates, in a schematic side view, the floating course of these threads 64 through the tunnel 38.

These further threads 64 running in the longitudinal direction are substantially electrically non-conducting, and they have a second water absorbency, which is higher than the first water absorbency of the threads 56, 58 of the second layer 36. The second water absorbency is preferably significantly higher than the first water absorbency of the non-conductive threads 56, 58 of the second layer 36. In particular, it is found to be advantageous if the second water absorbency of the threads 64 makes these threads appear hydrophilic and if the first water absorbency of the threads 56, 58 of the second layer 36 makes this layer appear hydrophobic. The water absorbency of threads can be determined in the manner already indicated at the beginning.

The hydrophilic threads 64 having a relatively higher second water absorbency and extended in the longitudinal direction through the entire flat textile material 2 are so provided and guided in the intermediate portion 32 in the case of the textile carrier layer 42 in the middle region 44 mentioned at the beginning that they, for example as warp threads of the textile carrier layer 42, repeatedly come into direct contact with the surface of the body. In this manner, the water- and moisture-absorbing threads 64 can keep a region of the surface of the body between the electrodes drier, which assists with the electrical insulation of the electrodes from one another. However, because the water- or moisture-absorbing threads 64 run spaced apart from the surface of the body in the electrode portion 30, that is to say in a floating manner in the region of the tunnel 38, that is to say on the side of the electrode 20 facing away from the body, they do not lead to drying out of the surface of the body in the region of direct contact with the electrodes 20. On the other hand, it is assumed that the water- or moisture-absorbing threads 64 can nevertheless carry out a certain moisture conditioning in the electrode portion 30, specifically in the region of the electrode 20. They protect the electrode 20 on the one hand from excess wetting and on the other hand from drying out by providing a moisture reservoir in the form of the moisture stored therein. Because, on the other hand, the electrically non-conductive threads 54 and 56 of the second layer 36 facing away from the body have a lower water absorbency than the threads 64 floating in the electrode portion 30 which have just been described, they in turn form protection toward the outside against drying out of the electrode 20 by evaporation and protection against the introduction of excessive water or moisture from outside to the electrode 20. It is thus considered to be particularly advantageous that the second layer 36 facing away from the body forms an outer water-repellent cover of the electrode 20 in the electrode portion 30 of the flat textile material 2. In particular in conjunction with water-attracting threads 64 provided on the side of the electrode 20 facing away from the body, the achievement of good contacting, that is to say of low contact resistance between the electrode 20 and the skin surface can be assisted.

Finally, the floating course of the electrically conductive threads 50 extended in the longitudinal direction in the intermediate portion 32 of the flat textile material will be discussed further. As already mentioned, these threads 50, together with the electrically conductive threads 52 extended in the transverse direction, form the electrode 20. Because the conductive threads 50 extended in the longitudinal direction, like preferably all the other threads extended in the longitudinal direction, are introduced endlessly in the manufacture of the flat textile material, they also extend between two electrode portions 30, that is to say in the intermediate portion 32, during manufacture. However, they extend there in a floating manner, as is shown schematically in FIG. 5 and in FIG. 2 on the left. This would connect the successive electrodes 20 together in an electrically conducting manner, which for example in some cases may be wholly desirable. If, on the other hand, this is not desirable, then it is possible to cut these floating threads 50, as is indicated in FIG. 2 at reference numeral 66. In this manner, the electrically conductive threads 50 can be cut close to the electrode portion 30 and the length that is produced can be discarded. It is, however, also conceivable and advantageous for the use of the flat textile material 2 or of a flat material portion 16 if the conductive threads 50 extended in the longitudinal direction are cut not close to the electrode but approximately centrally in the longitudinal direction, because the remaining lengths of the threads 50 can then be combined to form a bundle 70 (indicated in FIG. 6), which can be used as a contacting wire of the electrode 20. This bundle 70 can then additionally advantageously be grasped on the side of the flat material 2 facing away from the body and can be connected to line means leading from there to a measuring or treatment apparatus of any kind (which, however, is not shown).

FIGS. 7 and 8 show purely exemplary applications in the form of arrangements of a portion 16, in particular configured as described above, of a flat textile material 2 according to the invention on the human body. The flat material can be a wound dressing 72, in particular a plaster.

FIG. 8 shows a flat textile material 2 which is extended continuously in a head circumferential direction and closed on itself, which is thus joined to form a cylindrical portion 16 and comprises a plurality of electrode portions 30 having electrodes 20 and intermediate portions 32 arranged therebetween.

1. A flat textile material (2) for placing on a human or animal body to capture and/or transmit electrical signals, comprising:
a longitudinal direction (4),
a transverse direction (6) running transverse to the longitudinal direction (4),
an extension plane (8),
a thickness direction (10) orthogonal to the extension plane (8),
a first layer (34) facing the body,
a second layer (36) facing away from the body, and
at least one electrode (20) arranged on or in the first layer (34) for placing on the body, wherein
the electrode (20) comprises electrically conductive threads (50, 52),
the second layer (36) comprises electrically non-conductive threads (56, 58) having a first water absorbency,
there are provided either in the first layer (34) or in the thickness direction (10) between the electrode (20) and the second layer (36) threads (64) having a second water absorbency extending in the longitudinal direction (4), and
the second water absorbency of these threads (64) is greater than the first water absorbency of the electrically non-conductive threads (56, 58) of the second layer (36).

2. The flat material as claimed in claim 1, wherein the second water absorbency is at least 1.10 times, in particular at least 2.0 times, further in particular at least 5.0 times, even further in particular at least 10 times, and most particular at least 20 times, the first water absorbency.

3. The flat material as claimed in claim 1, wherein the first water absorbency is not more than 1.0 wt. %, in particular not more than 0.1 wt. %, and most particular not more than 0.01 wt. %.

4. The flat material as claimed in claim 1, wherein the second water absorbency is at least 2.0 wt. %, in particular at least 5.0 wt. %, and most particular at least 20 wt. %.

5. The flat material as claimed in claim 1, wherein the electrically conductive threads (50, 52) of the electrode (20) comprise electrically conductive threads (50) extending in the longitudinal direction (4) and electrically conductive threads (52) extending in the transverse direction (6).

6. The flat material as claimed in claim 1, wherein the electrically non-conductive threads (56, 58) of the second layer (36) comprise electrically non-conductive threads (56) extending in the longitudinal direction (4) and electrically non-conductive threads (58) extending in the transverse direction (6), which threads in each case have the first water absorbency.

7. The flat material as claimed in claim 1, wherein there is at least one side region (26) adjoining the electrode (20) in the transverse direction (6), in particular a first side region (26) adjoining the electrode (20) in the transverse direction (6) and a second side region (28) adjoining the electrode (20) in the transverse direction (6) opposite the first side region (26).

8. The flat material as claimed in claim 1, wherein the electrode (20) or the electrode (20) and the side region(s) (26, 28) form an electrode portion (30) of the flat material (2), and the electrode portion (30) is adjoined in the longitudinal direction (4) by an intermediate portion (32) of the flat material (2).

9. The flat material as claimed in claim 8, wherein the electrode portion (30) is adjoined in the longitudinal direction (4) by a further intermediate portion (32) opposite the intermediate portion (32).

10. The flat material as claimed in claim 8, wherein the intermediate portion (32) is adjoined in the longitudinal direction (4) by a further electrode portion (30), and the intermediate portion (3) is then arranged in the longitudinal direction between the electrode portions (30).

11. The flat material as claimed in claim 8, configured to be substantially endless in the longitudinal direction (4), wherein an electrode portion (30) and an intermediate portion (32) alternate.

12. The flat material as claimed in claim 1, wherein the first layer (34) comprises in the side region(s) (26, 28) of the electrode portion (30), electrically non-conductive threads (54) extending in the longitudinal direction (4) and electrically conductive threads (52) extending in the transverse direction (6).

13. The flat material as claimed in claim 1, wherein the threads (64) having the second water absorbency extending in the longitudinal direction (4) are arranged in the transverse direction (6) between the first side region (26) and the second side region (28).

14. The flat material as claimed in claim 13, wherein the threads (64) having the second absorbency are provided in a floating manner between the electrode (20) and the second layer (36).

15. The flat material as claimed in claim 1, wherein the first layer (34) and the second layer (36) lie loosely against or on one another at least in the electrode portion (30).

16. The flat material as claimed in claim 8, wherein the first layer (34) and the second layer (36) are joined together in the intermediate portion (32).

17. The flat material as claimed in claim 8, wherein the first layer (34) and the second layer (36) are brought together in the intermediate portion (32) to form a combined textile carrier layer (42).

18. The flat material as claimed in claim 1, wherein the threads (64) having the second water absorbency in the intermediate portion (32) are configured and arranged to be placed on the body.

19. The flat material as claimed in claim 8, wherein the electrically conductive threads (50, 52) of the electrode (20) arranged in the longitudinal direction (4) pass through the second layer (36) and/or the carrier layer (42) in the thickness direction (10), and are arranged in the intermediate portion (32) in a floating manner on the side of the second layer (36) or carrier layer (42) facing away from the body.

20. The flat material as claimed in claim 19, wherein the electrically conductive threads (50) arranged in a floating manner on the side of the second layer (36) or carrier layer (42) facing away from the body, are cut and in particular combined to form at least one bundle (70).

21. The flat material as claimed in claim 8, wherein the intermediate portion (32) comprises a middle region (44), the middle region (44) has a transverse extent in the transverse direction (6) that corresponds substantially to the electrode (20), there are provided in the middle region (44) threads (64) having the second water absorbency which extend in the longitudinal direction (4) and are arranged side by side substantially in parallel, and electrically non-conductive threads (56) having the first water absorbency which extend in the longitudinal direction and are arranged side by side substantially in parallel.

22. The flat material as claimed in claim 1, wherein, in the transverse direction (6) outside a middle region (44), the intermediate portion (32) is formed by electrically non-conductive threads (56) extending in the longitudinal direction (4) and electrically conductive and/or electrically non-conductive threads (58) extending in the transverse direction (6).

23. The flat material as claimed in claim 8, wherein the first layer (34) and the second layer (36) are arranged and joined together such that there is formed in the electrode portion (30), a tunnel (38) which extends in the transverse direction (6) and is delimited by the first and second layers (34, 36).

24. The flat material as claimed in claim 23, wherein a pressure element (40) can be inserted into the tunnel (38), which pressure element urges the first layer (34) and the electrode, and optionally the threads (64) having the second water absorbency which are guided transversely through the tunnel in a floating manner, toward the body.

25. The flat material as claimed in claim 5, wherein the electrode (20) is configured to be raised relative to the body compared to the side regions (26, 28), and/or the intermediate portion (32), and/or the carrier layer (42).

26. The flat material as claimed in claim 1, wherein the first and/or the second layer (34, 36) is formed by a woven fabric, a warp direction of the woven fabric runs in the longitudinal direction (4), and a weft direction of the woven fabric runs in the transverse direction (6).

27. A wound dressing (72) for assisting and monitoring wound treatment, wherein the wound dressing comprises a flat material (2) as claimed in claim 1.
